SEQUENCE LISTING (3 sequences)

SEQ ID NO: 1 - protein, 706 aa, synthetic construct
EVQLQQSGPELVEPGTSVRISCKTSGYTFTEYTIHWVKQSHGKSLEWIGHINPSNGGNSYNQRFKGTATLTADKSSSTAYMELRRLTSDDSAVYYCARWGWVYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 2 - protein, 448 aa, synthetic construct
EVQLQQSGPELVEPGTSVRISCKTSGYTFTEYTIHWVKQSHGKSLEWIGHINPSNGGNSYNQRFKGTATLTADKSSSTAYMELRRLTSDDSAVYYCARWGWVYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3 - protein, 214 aa, synthetic construct
DIQMTQTTSSLSASLGDRVTISCSASQDISNYLDWYQQKPDGTVRLLIYYTSSLHSGVPSRFSGSGSGTAFSLTIINLEPEDVATYYCHQYSNLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0011434 A1
(43) Pub. Date: Jan. 9, 2025

(54) ASYMMETRIC BISPECIFIC ANTIBODY (UMG2/CD1A-CD3E) FOR THE IMMUNOLOGICAL TREATMENT OF CORTICAL-DERIVED CD1A-EXPRESSING T-CELL ACUTE LYMPHOBLASTIC LEUKEMIAS (T-ALL)

(71) Applicant: UNIVERSITA' DEGLI STUDI MAGNA GRAECIA DI CATANZARO, CATANZARO (CZ) (IT)

(72) Inventors: Pierfrancesco TASSONE, CATANZARO (CZ) (IT); Daniele CARACCIOLO, CATANZARO (CZ) (IT); Maria Teresa DI MARTINO, CATANZARO (CZ) (IT); Licia PENSABENE, CATANZARO (CZ) (IT); Pierosandro TAGLIAFERRI, CATANZARO (CZ) (IT)

(73) Assignee: UNIVERSITA' DEGLI STUDI MAGNA GRAECIA DI CATANZARO, CATANZARO (CZ) (IT)

(21) Appl. No.: 18/706,938

(22) PCT Filed: Oct. 28, 2022
(86) PCT No.: PCT/IB2022/060394

§ 371 Date: May 2, 2024

(30) Foreign Application Priority Data
Nov. 2, 2021 (IT) 102021000027929

(51) Int. Cl. C07K 16/28

(52) U.S. Cl. CPC C07K 16/2833; C07K 16/2809; C07K 2317/31; C07K 2317/622

(57) ABSTRACT

Asymmetric bispecific antibody (UMG2/CD1a-CD3E) for the immunological treatment of cortical-derived CD1a-expressing T-cell acute lymphoblastic leukemias (T-ALL), against which the antibody specifically recruit, activate and redirect normal T-cells. Also, the immunological treatment of cortical-derived CD1a-expressing T-cell acute lymphoblastic leukemias (T-ALL and/or for the immunological treatment of Langherans histiocytosis, in which the asymmetric bispecific antibody (UMG2/CD1a-CD3E) is administered to a patient in need thereof.

### FIELD

The present invention relates to an asymmetric bispecific antibody, directed towards cortical-derived T-ALL cancer cells expressing CD1a, capable of recruiting, activating and redirecting normal T cells against cancer cells.

### BACKGROUND

T-ALL is a hematologic neoplasm characterized by an aberrant proliferation of T cell progenitors starting from an intratimical differentiation step that leads to the progressive infiltration of the bone marrow and lymphoid organs and the spread of immature leukemic T cells in the peripheral blood.

T-ALL is a hematologic, rare, orphan and very aggressive neoplasm characterized by frequently unfavorable evolution and poor prognosis in patients relapsed/refractory to conventional treatments. While the progress of immunotherapy has greatly improved the treatment of acute B-cell lymphoblastic leukemia (B-ALL), the lack of antigens with restricted expression on tumor T cells, therefore suitable for a selective targeting of the same saving the normal T-compartment, has significantly hindered the development of new immunotherapy strategies towards this important clinical condition. It is therefore absolutely important to identify and use therapeutic targets that provide the rationale for the development of new therapeutic strategies.

### SUMMARY

The object of the present invention is to provide a new bispecific antibody suitable to activate and redirect cytotoxic T-cells (bispecific T cell engager, BTCE), by means of CD3□ binding CD1a which is expressed by about 40% of T-ALL cases.

According to the present invention, a new asymmetric bispecific antibody, BTCE, is made against T-ALL.

### BRIEF DESCRIPTION OF THE FIGURES

For a better understanding of the present invention, a preferred embodiment is now described, by way of non-limiting example only, with reference to the figures, in which:

FIGS. 1A-1D show the experimental results relating to UMG2 antibody binding reactivity (mAb), in particular FIG. 1A shows the reactivity of UMG2 on HEK293T cell lines that do not express CD1a transfected with a control vector (left) and on HEK293T/CD1a transfected with a plasmid encoding CD1a (right);

FIG. 1B shows the reactivity of UMG2 on patient-derived T-ALL cells;

FIG. 1C shows the reactivity of UMG2 on cortical-derived T-ALL cell lines;

FIG. 1D shows the binding of UMG2 on peripheral blood cells from healthy donors.

FIGS. 2A-2F show experimental data relating to the in vitro cytotoxic activity of the UMG2/CD1a-CD3ε BTCE construct; in particular, FIG. 2A shows on the left: structure of UMG2-CD3ε BTCE, on the right: targeting of CD3+ T cells towards CD1a+ T-ALL cells mediated by the UMG2/CD1a-CD3ε BTCE construct evaluated by immunofluorescence microscopy analysis;

FIG. 2B shows data relating to the evaluation of the dissociation constant (Kd) of UMG2/CD1a-CD3ε BTCE evaluated by titration experiments on cortical T-ALL cell lines and CD1a-expressing;

FIG. 2C shows the relative percentage (%) of induced cytotoxicity HEK293T cells transfected with an empty plasmid or with a plasmid encoding CD1a;

FIG. 2D shows the relative percentage (%) of cytotoxicity on cortical-derived T-ALL, CD1a+ and CD1a− cell lines;

FIG. 2E shows data of the relative percentage (%) of cytotoxicity against primary CD1a+T-ALL cells (derived from patients);

FIG. 2F shows the increase in the concentration of CD107a on co-cultured T cells in an ET ratio of 10 a 1 (10:1) both in T-ALL cell lines expressing UMG2 and primary cells derived from T-ALL patients;

FIG. 3 shows experimental data relating to the in vitro functional activation of T cells in a concentration-dependent manner of UMG2/CD1a-CD3ε BTCE, assessed by activation of early and late markers (respectively CD69 and CD25) on both CD4 and CD8 positive T cells, by release of enzymes and cytotoxic molecules (granulisin, perforin and granzyme), and production of cytokines (TNF-α, IFN-γ and IL-2) assessed by co-culture experiments with HPB-ALL (A), TALL-1 (B) and Jurkat (C) in the presence of increasing concentration (0.01-0.1-1 μg/ml) of UMG2/CD1a-CD3ε BTCE.

FIG. 4 shows the in vitro activity of T-cell-mediated UMG2/CD1a-CD3ε BTCE, in particular FIG. 4A shows the level of NFAT1 protein on peripheral mononuclear blood cells (PBMCs) co-cultured with T-ALL cells in the presence of UMG2/CD1a-CD3ε BTCE or vehicle as a control;

FIG. 4B shows the proliferation of T cells co-cultured with HPB-ALL, TALL-1 and Jurkat cells respectively, in the presence of UMG2/CD1a-CD3ε BTCE;

FIG. 4C shows the extent of cytotoxicity towards neoplastic cells by PBMCs, to which CD4 and CD8 expressing lymphocytes, co-cultured with T-ALL cells and increasing concentrations of UMG2/CD1a-CD3ε BTCE have been subtracted.

FIG. 4D shows the relative percentage (%) of induced cytotoxicity on neoplastic cells by PBMCs, subtracted from CD56-expressing lymphocytes, or enriched with positive CD56, and PBMCs in the presence of Fc fragment blocker, co-cultured with Jurkat CD1a-expressing T-ALL cells and increasing concentration (0.01-0.1-1 μg/ml) of the BTCE construct;

FIG. 5 shows experimental data relating to the in vivo activity of UMG2/CD1a-CD3ε BTCE; in particular FIG. 5A shows the evaluation of in vivo fluorescence in NSG immunosuppressed mice (NOD/SCID gamma, non-obese diabetic/severe combined immunodeficiency), inoculated subcutaneously with HPB-ALL cells and treated with two weekly intraperitoneal doses of the BTCE construct at the concentration of 0.1 mg/kg or 0.5 mg/kg, or with the vehicle (PBS), as control;

FIG. 5B shows the survival curves (Kaplan-Meier) of the treated mice compared to the control group (log-rank test, p<0.05);

FIG. 6 shows the combined treatment data for UMG2/CD1a-CD3ε BTCE and immune-checkpoint inhibitors (ICIs). FIG. 6A shows the expression of T-cell anti-tumor function exhaustion markers (PD-1, TIM3, and TIGIT) on CD4 and CD8 positive T cells co-cultured with CD1a-expressing T-ALL cells in the presence of the BTCE construct in the presence of nivolumab and/or avelumab, compared to treatment with the BTCE construct alone. FIG. 6B shows the relative percentage (%) of cytotoxicity on CD1a-expressing T-ALL cells co-cultured with PBMC in the presence of the BTCE construct alone, nivolumab alone, avelumab alone, BTCE construct plus nivolumab or avelumab.

### DETAILED DESCRIPTION

According to the invention, a new asymmetric bispecific antibody (UMG2/CD1a-CD3ε) BTCE capable of recruiting T cells against cortical-derived T-ALL cells has been developed. In particular, the construct generated is an asymmetric 2+1, with a bivalent bond towards CD1a and monovalent towards CD3ε, made using the “knobs into holes” technology and starting from the sequence of scFv of the UMG2 antibody. The latter derives from the homonymous hybridoma obtained by long-term culture and numerous subclonings of the parental hybridoma UN5, already characterized and clustered as anti-CD1a. The UMG2 hybridoma deposited on 17 Sep. 2021, access number assigned by the International Depository Authority PD21003, produces the UMG2 antibody which has, compared to the parental antibody, high affinity for the epitope and a slight difference in the pattern of reactivity towards the target. The UMG2 antibody is therefore novel and directed towards a unique epitope of CD1a, a glycoprotein highly expressed by T-ALL cells.

The Applicant has carried out experimental tests, some of which are shown in the figures, in which the in vitro activity of the UMG2/CD1a-CD3ε BTCE construct was evaluated on cancer cells co-cultured with peripheral blood mononuclear cells derived from healthy donors (PBMCs) in whole or depleted of CD4/CD8 lymphocytes or enriched with CD56 lymphocytes with an E:T ratio equal to 10:1. In vivo anti-tumor activity of UMG2/CD1a-CD3ε BTCE was assessed in a mouse NSG model reconstituted with human peripheral blood mononuclear cells (Hu-PBMC) and inoculated with T-ALL cells. In vivo tumor growth was assessed by fluorescence imaging.

The Applicant investigated the targeting of CD1a as a potential specific target for T-ALL. CD1a is a transmembrane glycoprotein expressed on ˜40% of cortical-derived T-ALL cases and on a population of cortical thymocytes but not on peripheral blood cells. On non-hematopoietic cells, only a subset of dendritic cells residing in the skin (Langerhans cells, LC) express the CD1a antigen.

In order to develop a therapy against T-ALL, the Applicant has developed, according to the present invention, a new CD3ε-monovalent BTCE construct that simultaneously binds a single CD1a epitope in order to recruit and trigger a potent T-cell-mediated anti-tumor response.

The in vitro and in vivo activity of this BTCE is reported below, supporting the translational value of CD1a targeting strategies as a new therapy for patients with this aggressive disease.

By long-term culture and several subcloning procedures, the UMG2 clone was selected from a murine hybridoma previously generated by the group of proponent researchers, which was subsequently characterized as an anti-CD1a mAb. The UMG2 antibody recognizes its epitope on CD1a, while the affinity against the combinatorial site is slightly improved compared to the original parental clone. This clone was sequenced for generation of a bi-specific construct with a CD3ε monovalent arm.

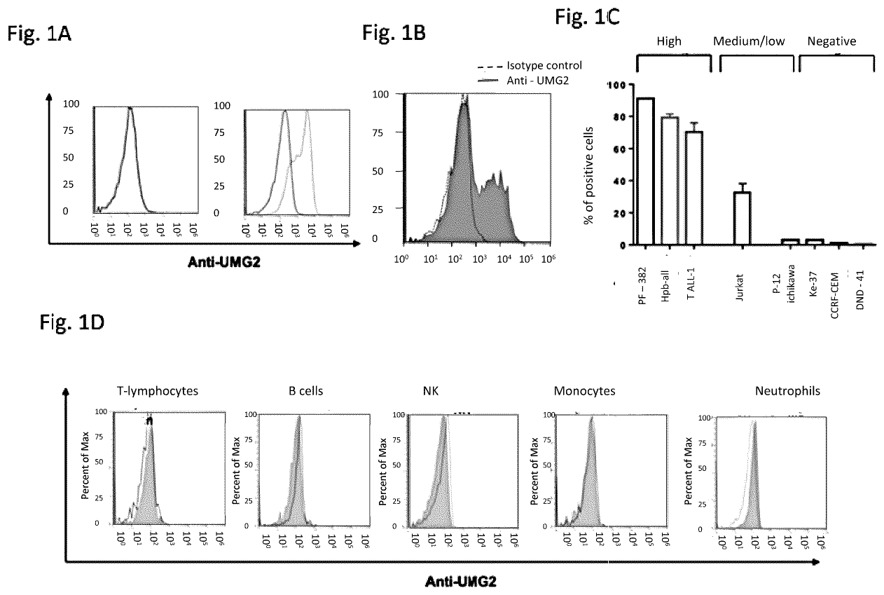

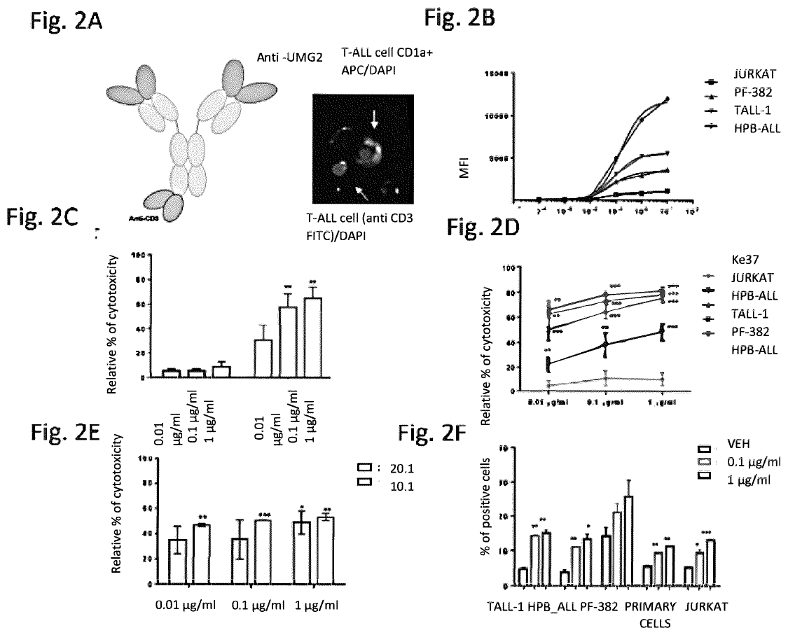

To confirm that UMG2 recognizes a CD1a epitope, the HEK293T cell line, negative for CD1a expression, was transfected with a plasmid encoding CD1a or with a negative empty vector (EV). After selection of the transfected clone, specific binding of UMG2 or anti-CD1a antibodies (SK9, BL6 HI149) to CD1a was assessed by flow cytometry. A strong binding of UMG2 was found on HEK293T cells expressing CD1a while no reactivity was observed on HEK293T cells transfected with the negative control (FIG. 1A). Importantly, a similar reactivity model was observed for the other anti-CD1a mAbs tested, further confirming the reactivity of UMG2 to CD1a (Complementary FIG. 1A-B). In addition, to investigate whether UMG2 binds to an original CD1a epitope, a competitive binding assay was performed between fluorochrome-conjugated UMG2 and fluorochrome SK9, BL6, and anti-CD1a HI149-conjugated mAbs. Interestingly, none of the anti-CD1a mAbs compete with the UMG2 binding (supplementary FIG. 2A), thus indicating that UMG2 recognizes a previously undescribed CD1a epitope.

Subsequently, the reactivity of UMG2 was evaluated on a panel of samples of T-ALL primary cells and cell lines. As shown in FIG. 1B, primary cortical T-ALL cells are recognized by UMG2. Among the 8 different cortical T-ALL cell lines tested, 3/8 are strongly positive for binding to labeled UMG2, 1/8 with lower intensity and 4 cell lines do not express the antigen (FIG. 1C). Interestingly, Ke-37, DND-41, and CCRF-CEM non-reactive cell lines with UMG2 are instead highly reactive to anti-CD1a antibodies SK9, BL6, and HI149, indicating once again that the epitope recognized by UMG2 is unique and characterized by a narrow pattern of expression (Supplementary FIG. 2B) compared to other anti-CD1a mAbs.

Finally, the reactivity of UMG2 on peripheral blood cells from healthy donors was evaluated. No reactivity was found on the different subtypes of blood cells (T cells, B lymphocytes, NK, monocytes and neutrophils) as expected (FIG. 1D), confirming a restricted expression pattern of the recognized epitope.

The structural characteristics of the asymmetric UMG2/CD1a-CD3ε BTCE, monovalent for the CD3□ arm (2+1), were developed to reduce the non-specific activation of T cells in the absence of a concomitant CD1a binding, while the CD1a reactivity domain was designed with a bivalent arm to enhance construct avidity for CD1a-expressing cells (FIG. 2A). To this end, then (2+1) UMG2/CD1a-CD3ε (clone L2K-07) BTCE was generated.

To define the apparent dissociation constant (Kd) of the new BTCE construct, titration experiments were performed. The average apparent Kd was estimated at 0.014 μg/mL, while binding saturation was achieved at concentrations of about 1 μg/mL (FIG. 2B).

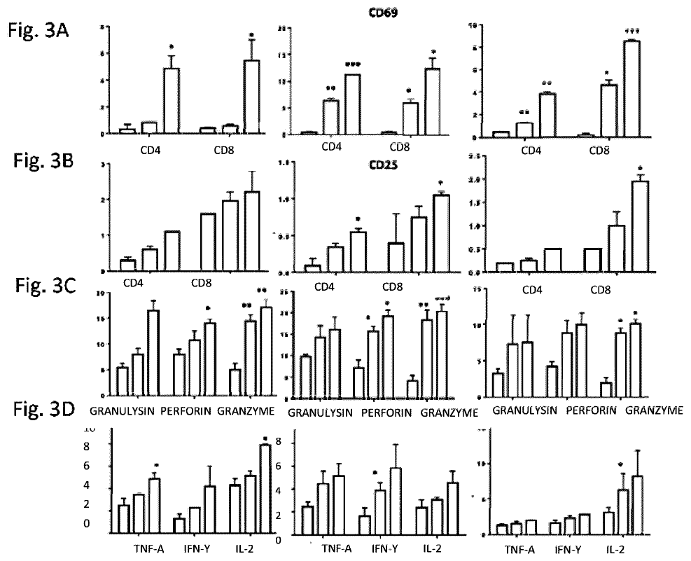

The in vitro activity of the BTCE construct was first evaluated using HEK293T/EV and HEK293T/CD1a cells, cultured together with PBMC as effector cells with effector/target ratio (E:T) 10:1. Concentration-dependent T-cell cytotoxicity was observed to HEK293T/CD1a cells but not to HEK293T/EV (FIG. 2C). To investigate whether BTCE induces a direct cytotoxic effect in the absence of T cells, the viability of HEK293T/CD1a and HEK293T/EV cells was assessed after 72 hours of treatment with the BTCE construct alone. No difference in cell viability was observed between HEK293T/CD1a and HEK293T/EV cells (Supplementary FIG. 3A).

To validate the translational relevance of these results, CD1a-expressing T-ALL cells (Jurkat, HPB-ALL, TALL-1, and PF-382) or non-expressing cells (Ke-37), were co-cultured with PBMC with an ET ratio of 10:1 in the presence of increasing BTCE concentration. Treatment resulted in a strong cytotoxic effect against CD1a-expressing T-ALL cells, while no significant cytotoxicity of T cells against CD1a-negative Ke-37 cells was observed (FIG. 2D). On primary cells from T-ALL-carrying patients, a cytotoxic effect equal to 60% of CD1a-expressing cancer cells was observed (FIG. 2E). Again, no direct cytotoxicity was observed in the absence of effector cells (Complementary FIG. 3B) and treatment with the BTCE construct does not trigger a direct cytotoxic effect. In addition, CD107a expression assessed in a concentration-dependent manner of the BTCE construct is increased in co-cultured T cells, with an E:T ratio of 10:1, with both CD1a-expressing T-ALL cell lines and with primary T-ALL cells, indicating cytotoxic degranulation induced by the BTCE construct (FIG. 2F).

To demonstrate activation of BTCE-guided T cells against CD1a-expressing T-ALL leukemic cells, high expression (HPB-ALL and TALL-1) and medium CD1a (Jurkat) T-ALL cell lines were co-cultured with PBMC with an E:T ratio of 10:1 in the presence of increasing concentration of the BTCE construct.

Importantly, UMG2/CD1a-CD3ε BTCE determines concentration-dependent T cell activation, as assessed by up-regulation of early and late activation markers CD69 and CD25 on both CD4/CD8 T cells, respectively, as well as by the release of granzyme, perforin, and granulysin, and the production of pro-inflammatory cytokines, such as TNF-α, IFN-γ, and IL-2 (FIG. 3A-3 D).

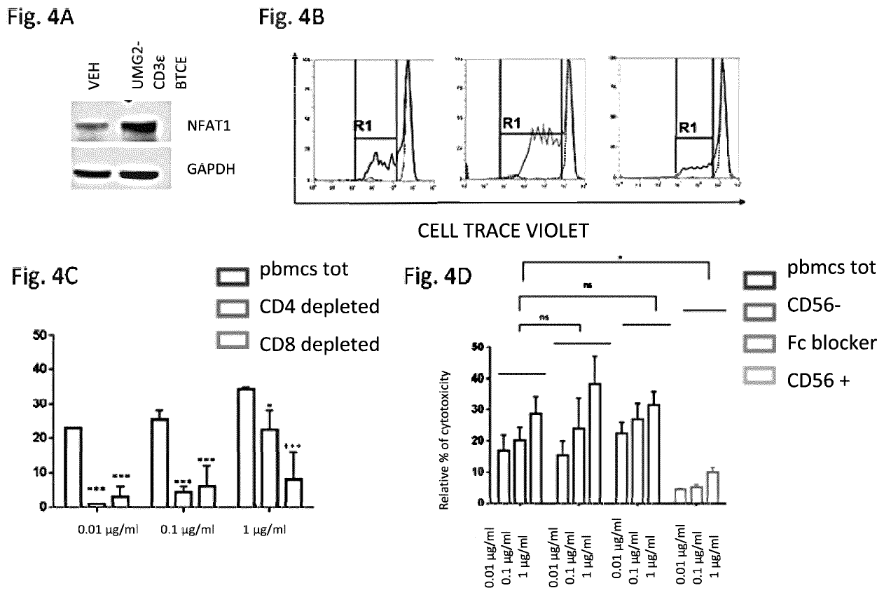

The CD3ε downstream signaling pathway was explored to investigate T cell activation. In particular, NFAT1 protein levels in PBMCs co-cultured at a 10:1 E:T ratio with CD1a-expressing T-ALL cell lines were evaluated, in the presence of the BTCE construct or vehicle as a control. Treatment with the BTCE construct induced higher levels of NFAT1 protein than control (FIG. 4A), inducing T cell proliferation (FIG. 4B).

Finally, to demonstrate that the cytotoxic effect induced by UMG2/CD1a-CD3ε BTCE is indeed dependent on T cells, CD1a-expressing T-ALL cells were exposed to an increasing concentration of UMG2/CD1a-CD3ε BTCE and co-cultured with PBMCs in their entirety, or PBMCs deprived of the fraction of CD4 or CD8-expressing T cells. Importantly, minimal cytotoxic activity was observed in both private CD4 or CD8 positive T cells samples compared to PBMCs in total (FIG. 4C). In addition, to exclude the presence of the Fc domain in the BTCE construct from inducing recruitment of natural immunity effector cells through the Fc-FcγR interaction by inducing cytokine release syndrome, the mechanisms of action of the BTCE construct by employing an Fc fragment blocker were further investigated. Jurkat cells expressing CD1a were co-cultured with an E:T ratio of 10:1 with either total PBMCs, or devoid of CD56-expressing T cells, or total PBMCs exposed to Fc blockers, or, finally, only CD56+ T cells. Considering that the minimal activity was found in the presence of only CD56+ T cells, both CD56+ T cell deprived PBMCs and PBMCs in total with blocked Fc, were able to achieve a cytotoxic activity comparable to the total PBMCs not depleted by the CD56+ component (FIG. 4D).

Together these results indicate that the BTCE construct exerts T-cell-mediated cytotoxicity against CD1a-expressing T-ALL cells that does not involve the activation of other cytotoxic cells.

Subsequently, the Applicant investigated the in vivo anti-tumor activity of the BTCE construct against CD1a-expressing T-ALL cells. Fluorescent HPB-ALL cells were inoculated subcutaneously in immunocompromised NSG mice. Seven days after subcutaneous inoculation of HPB-ALL cells, human PBMCs derived from a healthy donor were inoculated into the blood for the purpose of reconstituting human cytotoxic effects in experimental animals. Then, three days after PBMC engraftment, mice were randomized to receive intraperitoneal BTCE construct at two administrations per week. Tumor growth was assessed with an in vivo imaging system that detects tumor cell fluorescence.

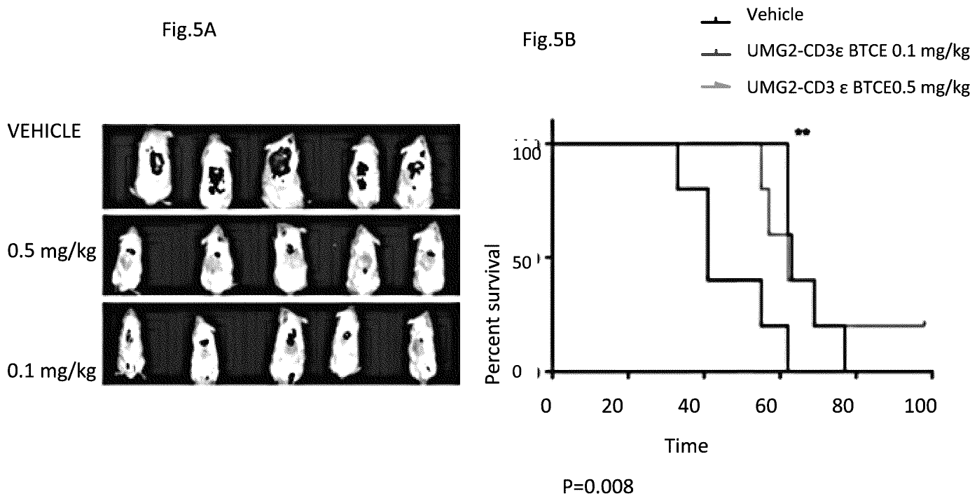

Treated animals were divided into two groups to receive the 0.1 or 0.5 mg/kg dose of the BTCE construct, and both experimental groups showed a significant reduction in tumor growth at both doses (FIG. 5A). This effect resulted in increased survival of animals in the treated groups. Precisely, a median survival of 41 days was evaluated in the vehicle-only group while 62 and 63 days was the median survival in the animal groups receiving 0.1 mg/kg and 0.5 mg/kg of the BTCE construct, respectively (FIG. 5B).

It is known that chronic antigenic stimulation induces overexpression of PDL-1/PDL-2 immune-checkpoints on cancer cells and expression of T-function depletion markers, such as PD-1, TIM-3, LAG-3 and TIGIT on T cells, thereby compromising the anti-tumor response in vivo. The Applicant investigated that immune-checkpoint inhibitors used in combination with the BTCE construct can counteract T cell dysfunction by representing a promising field of investigation.

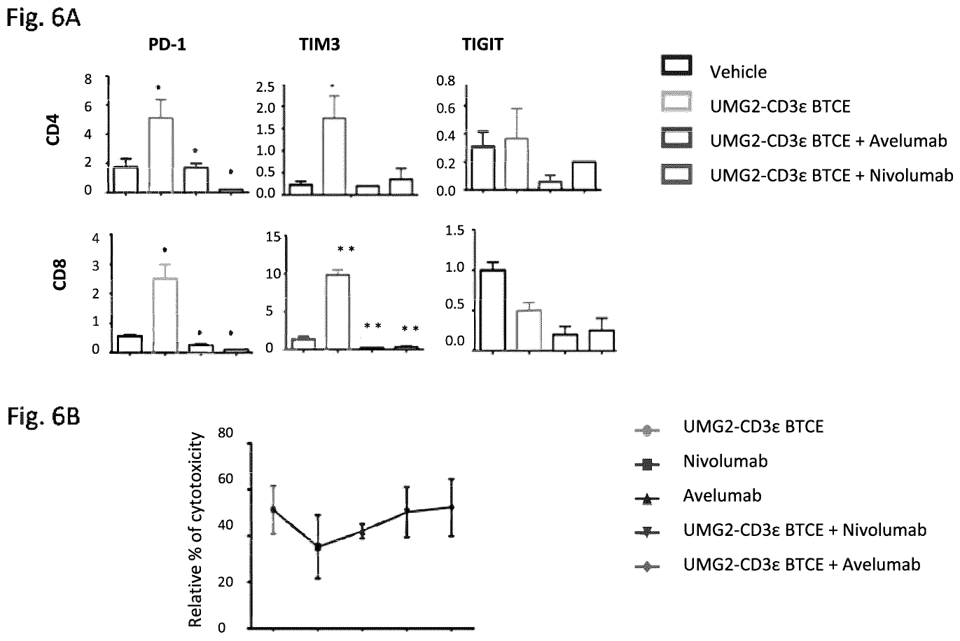

To assess whether chronic antigen stimulation induces T cell dysfunction on T cells, PBMC cells and labeled cells were co-cultured in the presence of the BTCE construct or vehicle, as a control. After 72 hours, T cells were again stimulated with 10:1 E:T-labeled T-ALL cells in the presence of the BTCE construct, or anti-PD-1, or anti PDL1 alone or in combination or the vehicle (control). A significant reduction in T cell depletion markers was found on T cells co-cultured with CD1a expressing cells treated with the combination of anti-PD-1/anti-PDL-1 and the BTCE construct, compared to BTCE alone (FIG. 6A).

To date, the treatment of patients with refractory/relapsing T-ALLs is still an objective to be achieved. Although immunotherapeutic approaches have revolutionized the prospects of patients with B-ALL, no immunotherapy-based strategy is currently approved and available for the treatment of T-ALL.

BTCE constructs represent an emerging approach to cancer immunotherapy, which is based on the promotion of immune synapses between effector T cells and tumor target cells, by antibody binding domains for both tumor-associated and T-cell-specific antigens. Thus, BTCE technology provides a novel functionality that is not present in any combination of parental antibodies.

There are several formats of BTCE constructs, ranging from very small proteins, consisting of two single-chain variable fragments (fragment-based), to larger immunoglobulin G (IgG)-like molecules.

According to the invention a construct formed from the structure of the UMG2 antibody, IgG2A with two CD1a binding arms, with a single scFv, derived from an anti-CD3 mAb OKT3 has been developed. This structure (asymmetric, 2+1) was chosen based on advantageous pharmacodynamic and pharmacokinetic considerations. First, the presence of bivalent binding domains for CD1a can increase selective recognition and elimination of cortical T-ALL cells highly expressing the CD1a antigen, saving healthy cells expressing it at low levels. Second, the monovalent arm for CD3 is preferred to the bivalent binding to avoid non-specific activation of T cells by CD3e to minimize entrapment of the BTCE construct in normal tissues rich in T lymphocytes. Third, the presence of the neonatal Fc receptor (FcRn), which protects the IgG-like BTCE from degradation, confers a long plasma half-life (days) compared to the shorter plasma half-life of the fragment-based BTCE (hours), which therefore must be continuously infused. In particular, in vitro, BTCE produced concentration-dependent T-cell activation, the release of inflammatory cytokines, and the induction of T-cell proliferation and activation, leading to lysis of T-ALL cells with an E:T-dependent ratio. Consistent with in vitro data, BTCE led to significant antitumor activity and survival benefit in animals treated in the in vivo trial. Importantly, for the translational value of the BTCE construct, the Applicant found this activity in a concentration range comparable to the doses used for the first already known and clinically approved BTCE, blinatumomab.

For what has been described sofar, the invention provides a potential new immunotherapeutic strategy for the treatment of T-ALL. Furthermore, BTCE-based therapy is an effective standard strategy that does not require the complex and expensive ex-vivo manipulation of effector cells as in the case of CAR-T for the re-direction of cellular immunity on the tumor. Finally, BTCEs are characterised by improved dose management, which reduces the risks associated with cytokine release syndrome (CRS) and other toxicities commonly associated with CAR-T therapy.

The experimental data obtained according to the invention support the feasibility and efficacy of a new BTCE targeted to T-ALL cells, with a very low potential risk of immunosuppression, supporting the concept of precision immunotherapy in these patients.

The UMG2/CD1a-CD3ε BTCE have been analyzed and sequenced. Hereby reported some results of the analytical results:

NAME bispecific mAb-CD3 (UMG2)

QUANTITY (mg) 42

TARGET (mg) 20

BONUS (mg) 22

CONC. (mg/ml) 4.63

VOLUME (ml) 9

BUFFER PBS 100 mmol/l L-arginine

ENDOTOXIN (EU/mg)<1 EU/mg

MONOMERICITY (%) 35.9%

OD 8.2

EXT. COEFF. 1.77

Sequences of UMG2 antibody are filed within this PCT application and are:

>PRO 39573_evi-5
UMG.2.VH3-h1v2.HC.S354C.T366W-anti.CD3.scFv
EVQLQQSGPELVEPGTSVRISCKTSGYTFTEYTIHWVKQSHGKSLEWIG
HINPSNGGNSYNQRFKGTATLTADKSSSTAYMELRRLTSDDSAVYYCAR
WGWVYALDYWGQGTSVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPCRDELTKNQVSLWCLVKGFYPSDIAVEWESNGQPENNY
KTTPPVLDSDGSFFLYSKLTVDKSRWQQGNVFSCSVMHEALHNHYTQKS
LSLSPGKGGGGSGGGGSGGGGSDIKLQQSGAELARPGASVKMSCKTSGY
TFTRYTMHWVKQRPGQGLEWIGYINPSRGYTNYNQKFKDKATLTTDKSS
STAYMQLSSLTSEDSAVYYCARYYDDHYCLDYWGQGTTLTVSSVEGGSG
GSGGSGGSGGVDDIQLTQSPAIMSASPGEKVTMTCRASSSVSYMNWYQQ
KSGTSPKRWIYDTSKVASGVPYRFSGSGSGTSYSLTISSMEAEDAATYY
CQQWSSNPLTFGAGTKLELK
>PRO 39574_evi-5
UMG.2.VH3-h1v2.HC.Y349C.T366S.L368A.Y407V
EVQLQQSGPELVEPGTSVRISCKTSGYTFTEYTIHWVKQSHGKSLEWIG
HINPSNGGNSYNQRFKGTATLTADKSSSTAYMELRRLTSDDSAVYYCAR
WGWVYALDYWGQGTSVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVCTLPPSRDELTKNQVSLSCAVKGFYPSDIAVEWESNGQPENNY
KTTPPVLDSDGSFFLVSKLTVDKSRWQQGNVFSCSVMHEALHNHYTQKS
LSLSPGK
>PRO 39575_evi-5 UMG.2.VL-hk(LEIK).LC
DIQMTQTTSSLSASLGDRVTISCSASQDISNYLDWYQQKPDGTVRLLIY
YTSSLHSGVPSRFSGSGSGTAFSLTIINLEPEDVATYYCHQYSNLPYTF
GGGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

In conclusion, it has been shown that the invention of the UMG2/CD1a-CD3ε BTCE construct could represent a safe and effective anti-T-ALL strategy to be investigated in a first-in-human clinical study as a maintenance treatment, like blinatumomab, for the elimination of minimal residual disease (MRD) or in refractory/relapsed patients expressing CD1a to improve the poor rate of disease control obtained with nelarabine. Furthermore, taking into account the good prognosis of patients with T-ALL expressing CD1a, the present invention can provide a framework for the incorporation of the new BTCE construct into a first-line chemo-free treatment as a bridge for allogeneic stem cell transplantation, thus opening up new opportunities for the treatment of T-ALL, which is still incurable today.

In addition, the asymmetric bispecific antibody (UMG2/CD1a-CD3 E) having the characteristics described is applicable to the immunological treatment of other pathological conditions such as Langerhans histiocytosis characterized by high expression of CD1a.

Finally, it is clear that the new asymmetric bispecific antibody (UMG2/CD1a-CD3ε) for the immunological treatment of pediatric and adult cortical-derived CD1a-expressing T-cell acute lymphoblastic leukemias (T-ALL) described and illustrated herein can be modified and varied without departing from the protective scope of the present invention, as defined in the attached claims.

1-7. (canceled)

8. An asymmetric bispecific antibody (UMG2/CD1a-CD3E) for the immunological treatment of cortical-derived CD1a-expressing T-cell acute lymphoblastic leukemias (T-ALL), against which said antibody specifically recruit, activate and redirect normal T-cells, said asymmetric bispecific antibody having sequence comprised in the group consisting of:
EVQLQQSGPELVEPGTSVRISCKTSGYTFTEYTIHWVKQSHGKSLEWIG
HINPSNGGNSYNQRFKGTATLTADKSSSTAYMELRRLTSDDSAVYYCAR
WGWVYALDYWGQGTSVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPCRDELTKNQVSLWCLVKGFYPSDIAVEWESNGQPENNY
KTTPPVLDSDGSFFLYSKLTVDKSRWQQGNVFSCSVMHEALHNHYTQKS
LSLSPGKGGGGSGGGGSGGGGSDIKLQQSGAELARPGASVKMSCKTSGY
TFTRYTMHWVKQRPGQGLEWIGYINPSRGYTNYNQKFKDKATLTTDKSS
STAYMQLSSLTSEDSAVYYCARYYDDHYCLDYWGQGTTLTVSSVEGGSG
GSGGSGGSGGVDDIQLTQSPAIMSASPGEKVTMTCRASSSVSYMNWYQQ
KSGTSPKRWIYDTSKVASGVPYRESGSGSGTSYSLTISSMEAEDAATYY
CQQWSSNPLTFGAGTKLELK,
EVQLQQSGPELVEPGTSVRISCKTSGYTFTEYTIHWVKQSHGKSLEWIG
HINPSNGGNSYNQRFKGTATLTADKSSSTAYMELRRLTSDDSAVYYCAR
WGWVYALDYWGQGTSVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVCTLPPSRDELTKNQVSLSCAVKGFYPSDIAVEWESNGQPENNY
KTTPPVLDSDGSFFLVSKLTVDKSRWQQGNVFSCSVMHEALHNHYTQKS
LSLSPGK,
DIQMTQTTSSLSASLGDRVTISCSASQDISNYLDWYQQKPDGTVRLLIY
YTSSLHSGVPSRFSGSGSGTAFSLTIINLEPEDVATYYCHQYSNLPYTF
GGGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

9. The asymmetric bispecific antibody according to claim 8, wherein said asymmetric bispecific antibody is a synthetic asymmetric 2+1 construct, formed by two scFv specific for CD1a (for a bivalent binding) derived from UMG2 antibody sequence bound to an IgG1 backbone in turn linked to a single scFv specific for CD3E (monovalent), derived from a mAb anti-CD3 OKT3.

10. The asymmetric bispecific antibody according to claim 8, wherein said asymmetric bispecific antibody is suitable to activate and to redirect cytotoxic T-cells, by means of CD3ε binding CD1a.

11. The asymmetric bispecific antibody according to claim 8, wherein said asymmetric bispecific antibody is an asymmetric 2+1 construct, having a bivalent binding to CD1a and a monovalent binding to CD3ε, generated using ‘Knobs-into-holes’technology and starting from UMG2 antibody development, directed against CD1a.

12. The asymmetric bispecific antibody (UMG2/CD1a-CD3E), wherein said asymmetric bispecific antibody is suitable for the immunological treatment of Langherans histiocytosis characterized by high CD1a expression.

13. A method for the immunological treatment of cortical-derived CD1a-expressing T-cell acute lymphoblastic leukemias (T-ALL) in a patient, comprising administering to the patient a therapeutically effective amount of the asymmetric bispecific antibody (UMG2/CD1a-CD3E) according claim 8.

14. Use of the asymmetric bispecific antibody (UMG2/CD1a-CD3E) according to claim 8, A method for the immunological treatment of Langherans histiocytosis in a patient, comprising administering to the patient a therapeutically effective amount of the asymmetric bispecific antibody (UMG2/CD1a-CD3E) according claim 8.
